(19) United States
(12) Patent Application Publication
(10) Pub. No.: US 2008/0033576 A1
(43) Pub. Date: Feb. 7, 2008

(54) X-RAY MARKER FOR MEDICAL IMPLANTS MADE OF A BIOCORRODIBLE METALLIC MATERIAL

(75) Inventors: Bodo Gerold, Zellingen (DE); Johannes Riedmueller, Nuernberg (DE); Heinz Mueller, Erlangen (DE); Claus Harder, Uttenreuth (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/834,719

(22) Filed: Aug. 7, 2007

(30) Foreign Application Priority Data
Aug. 7, 2006 (DE) 10 2006 038 238.2

(51) Int. Cl. A61F 2/00

(52) U.S. Cl. 623/23.75

(57) ABSTRACT

An x-ray marker for medical implants made of a biocorrodible metallic material, the x-ray marker comprises a boride or carbide of the elements tantalum or tungsten.

### PRIORITY CLAIM

This patent application claims priority to German Patent Application No. 10 2006 038 238.2, filed Aug. 7, 2006, the disclosure of which is incorporated herein by reference in its entirety.

### FIELD

The present disclosure relates to an x-ray marker for medical implants made of a biocorrodible metallic material, a medical implant having an x-ray marker, and a method for producing an x-ray marker for medical implants incorporating a boride or carbide of the elements tantalum or tungsten.

### BACKGROUND

Implants have found use in modern medical technology in manifold embodiments. Implants are used, for example, for supporting vessels, hollow organs, and duct systems (endovascular implants), for attaching and temporarily fixing tissue implants and tissue transplants, or even for orthopedic purposes, e.g., as nails, plates, or screws.

Frequently, only a temporary support or holding function until completion of the healing process or stabilization of the tissue is required and/or desired. In order to avoid complications which result from implants remaining permanently in the body, the implants must either be operatively removed or the implants must comprise a material which is gradually degraded in the body, i.e., a material that is biodegradable. The number of biodegradable materials based on polymers or alloys is continuously growing. For example, biocorrodible metal alloys made of magnesium, iron, and tungsten are known.

European Patent Application No. 1 270 023 describes a biodegradable magnesium alloy which is suitable for endovascular and orthopedic implants. The alloy may contain up to 5 weight-percent rare earths.

The biocorrodible metal alloys and polymers for medical implants known from the art have the disadvantage that the biocorrodible metal alloys and polymers are not visible or are not visible to a satisfactory extent in the current x-ray methods. However, x-ray diagnosis is an important instrument precisely for postoperative monitoring of the healing progress or for checking minimally invasive interventions. Thus, for example, stents have been placed in the coronary arteries during acute myocardial infarction treatment for some years. The stent is positioned in the area of the lesion of the coronary vascular wall and is intended to prevent obstruction of the vascular wall after expansion. The procedure of positioning and expanding the stent must be continuously monitored during the procedure by the cardiologist.

The x-ray visibility of an implant produced from a metallic or polymer material is the function, on one hand, of the material thickness and, on the other hand, of the x-ray absorption coefficient. The x-ray absorption coefficient is a function of the energy range of the x-ray radiation. In the medical field, the x-ray absorption coefficient is typically from 60 to 120 keV. The x-ray absorption coefficient typically becomes larger with rising atomic number in the periodic table and rising density of the material.

To improve the x-ray visibility, the implants are provided with markers, e.g., in the form of a coating, strip, an inlay, or a molded body made of a radiopaque material permanently bonded to the implant. Typically, the following points are also to be considered for the selection of the marker: (i) the functionality of the implant may not be restricted by the presence of the x-ray marker; (ii) the marker must be biocompatible; and (iii) the marker must be bonded to the implant in such a way that a loss thereof during implantation is precluded.

For implants which are designed to remain permanently in the body of the patient or are to be removed surgically at a later time, noble metals such as gold and platinum typically meet the cited criteria.

In implants made of biocorrodible metallic materials based on magnesium, iron, or tungsten, however, there are increased requirements for the marker material:
the marker is not to be detached prematurely from the main body of the implant by the corrosive processes, to avoid fragmentation and the danger of embolization;
the marker is to have sufficient x-ray density even at low material thicknesses, and
the marker material is to have no or at most a slight influence on the degradation of the main body.

German Patent Application No. 103 61 942 A1 describes a radiopaque marker for medical implants, which contains 10 to 90 weight-percent of a biocorrodible base component, in particular, from the group of elements consisting of magnesium, iron, and zinc. Furthermore, the marker contains 10 to 90 weight-percent of one or more radiopaque elements from the group consisting of I, Au, Ta, Y, Nb, Mo, Ru, Rh, Ba, La, Ce, Pr, Nd, Sm, Eu, Gd, Tb, Dy, Ho, Er, Tm, Yb, Lu, Hf, Ta, W, Re, Os, and Bi as a marker component. The markers described are suitable in principle for use in biocorrodible implants, in particular, those made of biocorrodible magnesium alloys.

However, a special problem arises upon the use of markers made of metallic materials on biocorrodible metallic main bodies where the degradation of the main body is altered in a contact area between marker and main body, i.e., the degradation of the main body is typically accelerated, because of electrochemical interactions between the two metallic materials.

### SUMMARY

The present disclosure provides several exemplary embodiments of the present invention.

One aspect of the present disclosure provides an x-ray marker for medical implants made of a biocorrodible metallic material, the x-ray marker comprising a boride or carbide of the elements tantalum or tungsten.

Another aspect of the present disclosure provides a medical implant, comprising an x-ray marker made of a biocorrodible metallic material, wherein the x-ray marker comprises a boride or carbide of the elements tantalum or tungsten.

A further aspect of the present disclosure provides a method for producing an x-ray marker for medical implants, comprising (a) forming an x-ray marker incorporating a boride or carbide of the elements tantalum or tungsten.

### DETAILED DESCRIPTION

For purposes of the present disclosure, the terms borides and carbides are collective names for compounds of boron and/or carbon respectively, with a metal, for example, tantalum or tungsten. The borides and carbides of tantalum and tungsten may be non-stoichiometric compounds having an alloy character.

The biocorrodible metallic material is preferably a biocorrodible alloy selected from the group consisting of magnesium, iron, and tungsten; in particular, the biocorrodible metallic material may be a magnesium alloy. For purposes of the present disclosure, an alloy is a metallic structure whose main component is magnesium, iron, or tungsten. The main component is the alloy component whose weight proportion in the alloy is highest. A proportion of the main component is preferably more than 50 weight-percent, in particular, more than 70 weight-percent.

If the material is a magnesium alloy, the material preferably contains yttrium and rare earth metals, because an alloy of this type is distinguished on the basis of the physiochemical properties and high biocompatibility, in particular, the degradation products.

A magnesium alloy of the composition rare earth metals 5.2-9.9 weight-percent, yttrium 3.7-5.5 weight-percent, and the remainder less than 1 weight-percent is especially preferable, magnesium making up the proportion of the alloy to 100 weight-percent. This magnesium alloy has already confirmed its special suitability experimentally and in initial clinical trials, i.e., the magnesium alloy displays a high biocompatibility, favorable processing properties, good mechanical characteristics, and corrosion behavior adequate for the intended uses. For purposes of the present disclosure, the collective term “rare earth metals” includes scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70), and lutetium (71).

The alloys of the elements magnesium, iron, or tungsten are to be selected in composition in such a way that the elements are biocorrodible. Artificial plasma, as has been previously described according to EN ISO 10993-15:2000 for biocorrosion assays (composition NaCl 6.8 g/l, CaCl2 0.2 g/l, KCl 0.4 g/l, MgSO4 0.1 g/l, NaHCO3 2.2 g/l, Na2HPO4 0.126 g/l, NaH2PO4 0.026 g/l), is used as a testing medium for testing the corrosion behavior of an alloy under consideration. A sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces by techniques known to those skilled in the art. The artificial plasma according to EN ISO 10993-15:2000 corresponds to a medium similar to blood and thus represents a possibility for reproducibly simulating a physiological environment.

A corrosion system comprises the corroding metallic material and a liquid corrosion medium, which simulates the conditions in a physiological environment in its composition or is a physiological medium, particularly blood. On the material side, factors, such as the composition and pretreatment of the alloy, microscopic and submicroscopic inhomogeneities, boundary zone properties, temperature and mechanical tension state, and in particular, the composition of a layer covering the surface, for example, influence the corrosion. On the side of the medium, the corrosion process is influenced by conductivity, temperature, temperature gradients, acidity, volume-surface ratio, concentration difference, and flow velocity.

For purposes of the present disclosure, implants are devices introduced into the body by a surgical method and comprise fasteners for bones, such as screws, plates, or nails, surgical suture material, intestinal clamps, vascular clips, prostheses in the area of the hard and soft tissue, and anchoring elements for electrodes, in particular, of pacemakers or defibrillators.

The implant is preferably a stent. Stents of typical construction have a filigree support structure made of metallic struts which is initially provided in an unexpanded state for introduction into the body and is then widened into an expanded state at the location of application.

According to a preferred exemplary embodiment, the x-ray marker is a tantalum carbide or a tungsten carbide, especially preferably TaC. The cited materials are distinguished by good x-ray visibility for medical use and inert behavior in relation to physiological media.

The x-ray marker may be provided in a solid embodiment as solid material and may be connected to the implant by suitable retention elements or by gluing, soldering, and stapling, for example. However, according to a preferred exemplary embodiment, the x-ray marker is provided as a powder having a mean particle size in the range from 0.1 to 20 μm; the powder is embedded in an organic biodegradable carrier matrix. The organic carrier matrix essentially comprises an organic compound, in particular, a polymer. The advantage is, inter alia, the simplification of the processing; a dispersion made of the two components of organic carrier matrix and x-ray marker powder, possibly with a suitable solvent added, may be produced, which may be applied to the implant via typical coating methods or may be used as a filler material for a cavity in the implant. After the degradation of the biocorrodible carrier matrix, the x-ray marker powder remains and is probably, but not necessarily, stored in extracellular vesicles because of the small particle size. It is to be assumed that an intercalation of the material of this type reduces rejection reactions.

According to a first exemplary variant, the biodegradable carrier matrix comprises one or more biodegradable polymers entirely or at least 80 weight-percent, in relation to the total weight of the carrier matrix. In particular, the carrier matrix comprises a polylactide, e.g., poly-L-lactide. According to a second exemplary variant, the biodegradable carrier matrix may comprise one or more biodegradable fats or oils entirely or at least 80 weight-percent, in relation to the total weight of the carrier matrix.

In a further exemplary embodiment having a powdered x-ray marker, which may particularly also be implemented using the two above-mentioned preferred variants of the biodegradable carrier matrix, a weight proportion of the x-ray marker in relation to the total weight of carrier matrix and x-ray marker is in the range from 15 to 90 weight-percent. In the first exemplary variant having a carrier matrix made of a biodegradable polymer, the weight proportion of the x-ray marker in relation to the total weight of carrier matrix and x-ray marker is preferably in the range from 15 to 90 weight-percent. In the second exemplary variant having an organic biodegradable carrier matrix made of a fat or oil, the weight proportion of the x-ray marker in relation to the total weight of carrier matrix and x-ray marker is preferably in the range from 80 to 90 weight-percent. It is thus ensured, on one hand, that the material has sufficient x-ray visibility even at relatively low material thicknesses and, on the other hand, processing as a dispersion is still possible.

A second exemplary embodiment provides a medical implant having an x-ray marker of the compositions described hereinabove. In particular, this medical implant is a stent, preferably a stent made of a biocorrodible magnesium alloy.

Finally, a third exemplary embodiment relates to the use of a boride or carbide of the elements tantalum or tungsten as an x-ray marker for medical implants. The present disclosure also provides a method for producing an x-ray marker using boride or carbide of the elements tantalum or tungsten produced by a method described herein.

A stent made of the biocorrodible magnesium alloy WE43 (93 weight-percent magnesium, 4 weight-percent yttrium [W], and 3 weight-percent rare earth metal [E]) was coated with an x-ray marker as described below.

### EXAMPLES

### Example 1

### TaC in Polymer Carrier Matrix

A dispersion made of a PEG/PLGA copolymer (diblock copolymer made of polyethylenglycol (PEG) and poly(DL-lactide-co-glycolide) (PLGA) with Mw 5,000; available from Boehringer Ingelheim, Germany, under the trade name Resomer RGP d 50155) and TaC-Pulver (available from Chempur), having a mean particle size of approximately 10 μm, was prepared in acetone, a weight proportion of the TaC powder in relation to the total weight of copolymer and x-ray marker being 75 weight-percent. The stent ends were immersed in the dispersion, which was homogenized by stirring, and subsequently dried in air.

The resulting droplets made of TaC/Resomer had a thickness of approximately 150 μm after multiple immersions.

Exemplary embodiment 2—TaC in a fat: 90 weight-percent TaC powder (reference source as in exemplary embodiment 1) was stirred into 10 weight-percent hydrogenated soybean oil at approximately 65° C. (both available from Hees) and was homogenized. This suspension was then dispersed into a cavity in the stent.

All patents, patent applications and publications referred to herein are incorporated by reference in their entirety.

What is claimed is:

1. An x-ray marker for medical implants made of a biocorrodible metallic material, the x-ray marker comprising a boride or carbide of the elements tantalum or tungsten.

2. The x-ray marker of claim 1, wherein the biocorrodible metallic material is an alloy selected from the group consisting of magnesium, iron, and tungsten.

3. The x-ray marker of claim 2, wherein the biocorrodible metallic material is a magnesium alloy.

4. The x-ray marker of claim 1, wherein the implant is a stent.

5. The x-ray marker of claim 1, wherein the x-ray marker is a tantalum carbide or tungsten carbide.

6. The x-ray marker of claim 1, wherein the x-ray marker comprises a powder having a mean particle size of the range of 0.1-20 μm and the powder is embedded in a biodegradable carrier matrix.

7. The x-ray marker of claim 6, wherein the carrier matrix comprises one or more biodegradable polymers entirely or at least 80 weight-percent, in relation to the total weight of the carrier matrix.

8. The carrier matrix of claim 6, wherein the carrier matrix comprises one more biodegradable fats or oils entirely or at least 80 weight-percent in relation to the total weight of the carrier matrix.

9. The x-ray marker of claim 6, wherein a weight proportion of the x-ray marker in relation to the total weight of the carrier matrix and the x-ray marker is in the range from 15 to 90 weight-percent.

10. A medical implant, comprising an x-ray marker made of a biocorrodible metallic material, wherein the x-ray marker comprises a boride or carbide of the elements tantalum or tungsten.

11. The medical implant of claim 10, wherein the biocorrodible metallic material is an alloy selected from the group consisting of magnesium, iron, and tungsten.

12. The medical implant of claim 11, wherein the biocorrodible metallic material is a magnesium alloy.

13. The medical implant of claim 10, wherein the x-ray marker is a tantalum carbide or tungsten carbide.

14. A method for producing an x-ray marker for medical implants, comprising:
(a) forming an x-ray marker incorporating a boride or carbide of the elements tantalum or tungsten.
